(19) United States
(12) Patent Application Publication
(10) Pub. No.: US 2021/0087602 A1
(43) Pub. Date: Mar. 25, 2021

(54) APPARATUS FOR SCREENING AND DIAGNOSIS OF MENINGITIS

(71) Applicant: Kalthom Adam Hassan IBRAHIM, Omdurman Abo Said, Khartoum (SD)

(72) Inventor: Kalthom Adam Hassan IBRAHIM, Omdurman Abo Said, Khartoum (SD)

(21) Appl. No.: 16/965,657

(22) PCT Filed: May 3, 2018
(86) PCT No.: PCT/SD2018/000005

§ 371 Date: Jul. 29, 2020

(30) Foreign Application Priority Data
Jan. 29, 2018 (SD) 4683/2018

(51) Int. Cl. C12Q 1/04; G16H 30/40; G16H 50/20

(52) U.S. Cl. CPC C12Q 1/04; G01N 2333/22; G16H 50/20; G16H 30/40

(57) ABSTRACT

The prior art of diagnosis of meningitis is based on the preparation of CSF and treated by Gram stain to show the shape of the bacteria by optical microscopy, then make a laboratory culture for the CSF to confirm the results as FIG. 2.

An apparatus for the diagnosis of meningitis comprises;
Power source;
Standard sliding glass;
Digital camera chip;
Microprocessor chip;
Software for image processing;
Software to analyze image features;
Programs for automated learning;
Digital screen;
The system of diagnosis meningitis uses image processing and automated learning. The methodology included five stages starting with the preparation of the CSF scan and dyeing it by the appropriate dye and digitizing it through a high-magnification scanning camera, then treated, segmenting and isolating the areas of interest by the image processing program, extracting the features and selecting the appropriate features for the classification, finally classification of meningitis.

### BACKGROUND

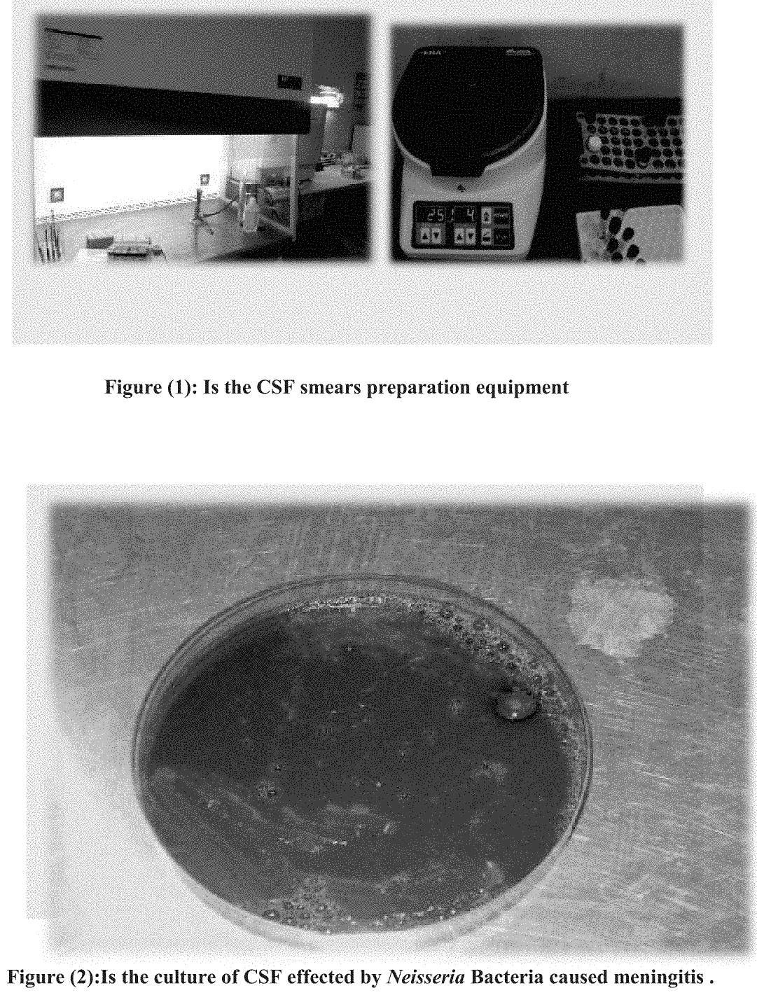

The prior art of diagnosis of meningitis is based on the preparation of CSF and treated by Gram stain to show the shape of the bacteria by optical microscopy, then make a laboratory culture for the CSF to confirm the results and determine the type of bacteria. FIG. 2 illustrate sample for microbiology culture infected by Bactria type Neisseria.

Medical diagnosis is a procedure for identifying a disease by critically analyzing its symptoms and assisted by the most complex series of laboratory tests of varying complexity. Accurate medical diagnosis is necessary in order to provide the most effective therapeutic option early and avoid the spread of the disease, leading to complications including permanent disability or death as in meningitis.

### DESCRIPTION

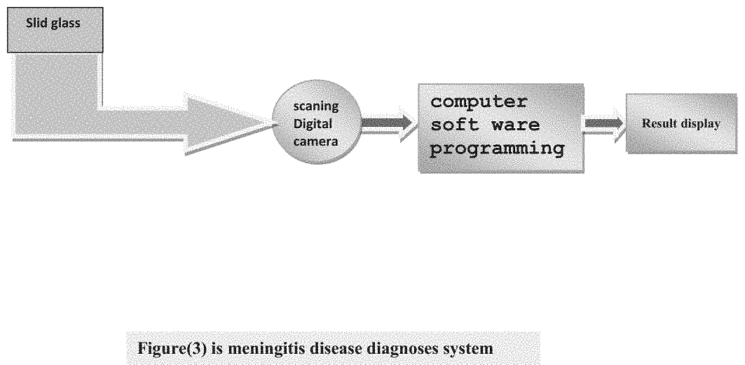

In an embodiment of the invention an apparatus for screening and diagnosis of meningitis disease comprising, a power source; a standard sliding glass; a high-magnification and scanning camera; a microprocessor chip; a programs for images processing techniques; a programs for analyses images features; a software for a machine learning techniques; a digital display; FIG. 3 is illustrate the sequence of the meningitis diagnoses system.

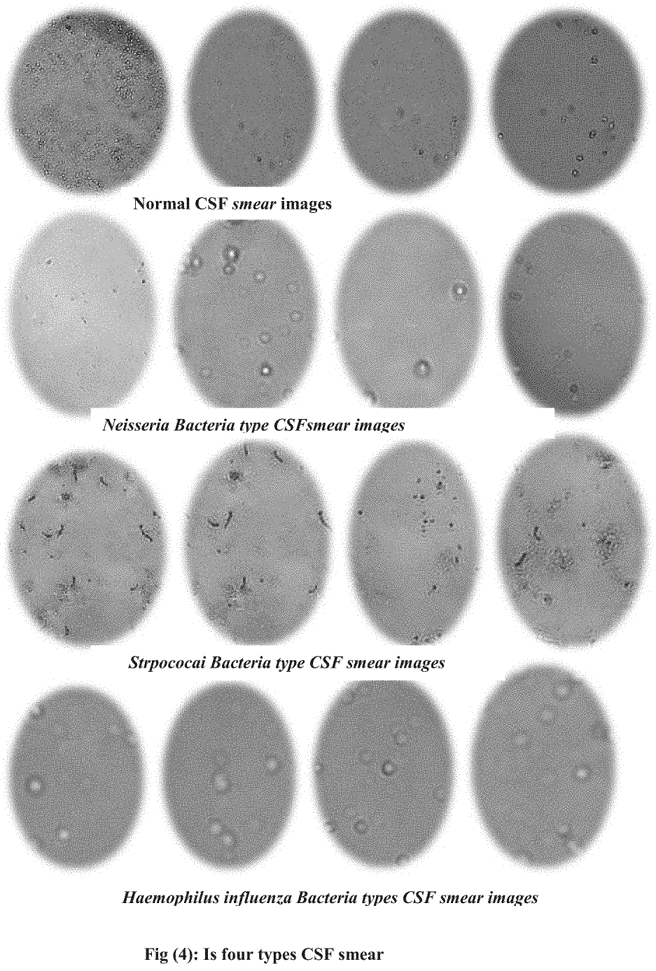

The meningitis disease diagnostic methodology is comprising, smear preparation, image processing techniques and machine learning techniques which are contained several phases represented on the preparation of CSF smear and treated by Gram stain, fig (l) is illustrate preparation equipment, then digitizing it through a high-magnification and scanning camera, FIG. 4 is illustrate the meningitis smear images.

The CSF stained images are first obtained as inputs to the diagnostic process and then analyzing through meningitis diagnostic phases, the images are segmented and the important area is isolated depending on the forms of bacteria causing the disease or the natural components of the cerebrospinal fluid. The properties of these images are derived according to the characteristics and features of the color, shapes and texture after conducting mathematical operations and programs for processing the digital images. Each type selected based on its features by apply machine learning techniques.

### Implementation Requirement:

The design and implementation of this invention includes two basic parts, namely, the development of many samples conform to the limit for the design of medical devices and testing, and the second part is the embodiment of the system software and hardware.

The first part of the implementation of the invention needs the care and guidance of the World Health Organization or government bodies have the powers to supervise the health centers because this part of the implementation needs to apply a protocol of health and also has the ethical considerations related to taking samples of patients suspected of infection.

The second part of the design and application of the invention is concerned by the companies specialized in the design and production of parts, namely hard ware and software. The first includes the design of power panels, camera chip, microprocessor chip and display. The software program includes digital image processing, analysis and extraction properties and features, and machine learning programs. These parts are integrated into one system that is compatible with each other. It is also compatible with the sample preparation method to produce a single product which is a correct, fast, reliable diagnosis for a suspected patient.

Invention can be implemented as an intelligent image based diagnostic system

The prior art of screening and diagnosis of meningitis is mainly based on the taking of a sample of cerebrospinal fluid (CSF) and its scanning on a glass slide where it. is treated with the appropriate dye to show the bacteria and then examined by the optical microscope. Laboratory culture of the (CSF) sample is also performed to confirm the results and determine the type of bacteria as in FIG. 2.

1: In the embodiment of the invention, An apparatus for screening and diagnosis of meningitis
disease, comprising:
A power source;
a standard slid glass;
a high-magnification and scanning camera;
a microprocessor chip;
a programs for images processing techniques;
a programs for analyses images features;
a programs for a machine learning techniques;
a digital display;

2: A method screening and diagnosis of meningitis disease, comprising:
A Preparing smear of cerebrospinal fluid and staining it by grain stain;
a digitizing of cerebrospinal fluid smears using high-magnification and scanning camera;
an isolating the image of Bacteria;
an applying Several image processing techniques to Bactria images;
a segmenting techniques for the Becteria images; an isolating of Bacteria images;
an extracting of images features;
a selecting of appropriate images features;
a Classifying of images features by a machine a ning techniques;
a detecting for normal images or infected;

3: Production and manufacturing protection includes the intermediate and final product. wherein, the intermediate and final products have the same basic structural elements, so that the material is technically interrelated. The final product is manufactured directly from a medium or separated by a small number of intermediate component, all of which contain the same basic structural element.
